(19) United States
(12) Patent Application Publication
(10) Pub. No.: US 2006/0194829 A1
(43) Pub. Date: Aug. 31, 2006

(54) Therapeutic materials and methods

(76) Inventors: Timothy P. Clackson, Arlington, MA (US); Camille L. Bedrosian, Belmont, MA (US)

Correspondence Address: CHOATE, HALL & STEWART LLP, TWO INTERNATIONAL PLACE, BOSTON, MA, 02110, US

(21) Appl. No.: 11/312,296

(22) Filed: Dec. 20, 2005

Related U.S. Application Data
(60) Provisional application No. 60/638,054, filed on Dec. 20, 2004.

(51) Int. Cl. A61K 31/4745; A61K 31/4545; A61K 31/445

(52) U.S. Cl. 514/291; 514/316; 514/317

(57) ABSTRACT

Disclosed are compositions and methods for treating or preventing mucositis by administering to the patient an FKBP ligand prior to, during, or after treatments commonly associated with the development of mucositis such as certain chemotherapies, radiation therapies, or combinations thereof, but in particular, administration of an mTOR inhibitor such as rapamycin, AP23573, CC1779 or everolimus.

### CROSS REFERENCE TO RELATED APPLICATIONS

This application claims priority to U.S. Provisional Patent Application Ser. No. 60/638,054 filed Dec. 20, 2004, the entirety of which is hereby incorporated herein by reference.

### TECHNICAL FIELD

This invention provides compositions and methods for treating or preventing mucositis.

### BACKGROUND

Mucositis is a significant side effect of cancer chemotherapy and radiation therapy, commonly appearing within 5 to 10 days of chemotherapy or radiation treatment and lasting for up to several weeks. The term “mucositis” encompasses mucosal damage throughout the alimentary tract, including, among others, oral, oropharyngeal and gastrointestinal mucositis. It can be terribly painful, can limit a patient's nutrient intake and/or absorption and can increase the length of hospital stay and the risk of serious infection. In addition, the onset of mucositis is often dose limiting with respect to the cancer treatment resulting in a less effective treatment for the patient. A wide variety of treatments for mucositis have been proposed and tried, but unfortunately this very serious problem persists. For a review of the pathobiology of mucositis, see Stephen T. Sonis, Nature Reviews | Cancer vol. 4, 277-284 (April 2004). See also, Rubenstein et al, Mucositis: Perspectives and Clinical Practice Guidelines Supplement to Cancer vol. 100, No. 9, pp. 2026-2046 (May 1, 2004). A new treatment to help prevent or treat mucositis would be of great benefit for cancer patients.

### SUMMARY OF THE INVENTION

This invention provides compositions containing an FKBP ligand for treating or preventing mucositis, especially mucositis associated with treatment of a patient with an mTOR inhibitor. The present invention therefore provides, among other things, certain pharmaceutical compositions, and methods of treating patients suffering from or susceptible to mucositis with such compositions.

### DETAILED DESCRIPTION OF CERTAIN EMBODIMENTS

Mucositis is a significant side effect of cancer chemotherapy and radiation therapy, commonly appearing within 5 to 10 days of drug or radiation treatment and lasting for up to several weeks. Mucositis can involve pain, redness, inflammation, ulceration, or combinations thereof, affecting the gastrointestinal tract from the mouth to anus. It usually results from disease or is secondary to therapeutic treatments such as certain chemotherapies, ionizing radiation, or combinations thereof, or to other mucositis-inducing circumstance and events. Specific forms of mucositis include, among others, oral mucositis, esophagitis, enterititis and colitis.

It has been observed that patients receiving an mTOR inhibitor develop a different type of mucositis. In these patients who have received an mTOR inhibitor, the mucositis often develops in 3 to 5 days rather than the 5 to 10 days as is typical among patients receiving conventional chemotherapies and radiation therapy. In addition, the mucositis which develops in patients receiving an mTOR inhibitor presents itself with oral lesions which differ from those associated with typical mucositis.

It has been observed that some patients receiving an mTOR inhibitor develop extremely painful lesions within the mouth and adjoining tissues. To health care providers, it is readily apparent that the time of onset of lesions after administration of the drug, the morphology of the lesions, their clinical course and presumably, their underlying pathobiological mechanism are dramatically different from mucositis associated with chemotherapy or radiation. Nonetheless, in this document we have adopted the oversimplification of referring to the lesions associated with the administration of an mTOR inhibitor as “mucositis” because others use this imprecise label for the mTOR inhibitor-associated syndrome, and a more precise label has not yet been adopted. Thus the reader should be aware that the accumulated knowledge concerning mucositis from other causes may not be particularly relevant or helpful in the painful and dose-limiting syndrome associated with administration of an mTOR inhibitor.

A new treatment to help prevent or treat “mucositis” associated with administration of an mTOR inhibitor would be of great benefit for those cancer patients.

According to one embodiment, the present invention provides a method for treating or preventing mucositis in a patient comprising administering to said patient an FKBP ligand. In certain embodiments, the FKBP ligand is administered topically or locally. Such administration may occur prior to the administration of an mTOR inhibitor, concurrently with the administration of an mTOR inhibitor, or after the administration of an mTOR inhibitor. Accordingly, it is contemplated that the present method is useful for both treating and preventing mucositis induced by the systemic administration of an mTOR inhibitor. In certain embodiments, the mTOR inhibitor is administered for the purpose of treating cancer in the patient. Such mTOR inhibitors are known to one of skill in the art and include, but are not limited to rapamycin or an analog or derivative thereof, e.g., temsirolimus (CCI-779, Wyeth), everolimus (RAD001, Novartis), ABT-578 (Abbott Labs) or AP23573 (ARIAD Pharmaceuticals, Inc.). The mTOR inhibitor may be administered for treating any of a variety of cancers, or for other well known applications of mTOR inhibitor s including the prevention of transplant rejection and others.

The following definitions and additional background information may be helpful to the reader.

The terms “oral mucositis” and “stomatitis” are sometimes used interchangeably and typically refer to lesions affecting any surface of the oral pharyngeal and/or laryngeal epithelial surface. The terms have been used loosely and imprecisely in some cases in the literature. “Esophagitis” typically refers to mucositis affecting the esophagus.

As used herein, the term “treating” refers to lessening the severity of mucositis or any symptoms and/or conditions related to mucositis. In certain embodiments, the term “treating” includes alleviating.

The term “alleviating” as use herein, unless otherwise specified, refers to preventing the occurrence of mucositis, decreasing the surface area of tissues that are affected by mucositis, reducing the intensity of mucositis, and/or enhancing or accelerating the rate at which these tissues heal and return to a normal or more normal state.

Numerical ranges in this document are intended to include every number and subset of numbers contained within that range, whether specifically disclosed or not. Moreover, these numerical ranges are intended to cover all included numbers and ranges within the recited range. For example, a disclosure of from I to 10 is intended to include a range of from 2 to 8, from 5 to 6, from 4.2 to 6.6, from 4.1 to 9.9, etc.

All references to singular characteristics or limitations are intended to include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination of steps is made.

All percentages, parts and ratios as used herein are by weight of the total composition, unless otherwise specified. All such weights as they pertain to listed ingredients are based on each respective ingredient per se and, therefore, do not include solvents or by-products that may be included in commercially available materials, unless otherwise specified.

The compositions and methods of the present invention can comprise additional or optional ingredients, components, or limitations described herein or otherwise useful in compositions and methods of the general type as described herein.

1. mTOR Inhibitors

As described above, methods of the present invention are useful for treating or preventing mucositis induced by the systemic administration of an mTOR inhibitor. Exemplary mTOR inhibitors include rapamycin or an analog or derivative thereof which form a complex with FKBP and mTOR. Rapamycin is a macrolide produced by Streptomyces hygroscopicus and discovered in the 1970's. Rapamycin is a potent immunosuppressive agent and is used clinically to prevent rejection of transplanted organs. It has also been reported to have activity as an antifungal agent, in the experimental allergic encephalomyelitis model (a model for multiple sclerosis), in the adjuvant arthritis model (for rheumatoid arthritis), and in inhibiting the formation of IgE-like antibodies. In addition, rapamycin has been reported to be useful for treating or preventing lupus erythematosus, pulmonary inflammation, insulin dependent diabetes mellitus, adult T-cell leukemia/lymphoma, smooth muscle cell proliferation and intimal thickening following vascular injury as well as certain cancers. See e.g. U.S. Pat. appln 2001/0010920. A number of derivatives of rapamycin, including AP23573 (ARIAD), CCI779 (“temsirolimus”, Wyeth) and RAD001 (“Everolimus”, Novartis) have yielded promising results in human studies involving a variety of cancers. In addition, rapamycin and everolimus are used as immunosuppressants in organ transplant recipients. Rapamycin and a number of the C-43-modified rapamycin analogs, including among others AP23573 and ABT-578 (a rapamycin derivative bearing a tetrazole moiety in place of the OH at position 43), are being used, evaluated or developed for use on stents as anti-restenotic agents following interventional cardiology.

Because there is more than one accepted convention for numbering the positions of rapamycin, and derivatives thereof, the numbering convention used herein is included in the depiction below for clarity.

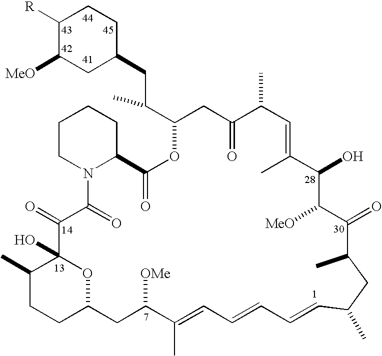

Certain rapamycin derivatives having substituents at the C-43 position are being developed as mTOR inhibitors. In certain representative compounds, R is —OH (rapamycin), —OP(O)(Me)₂ (AP23573), —OC(O)C(CH₃)(CH₂OH)₂ (temsirolimus), or —OCH₂CH₂OH (everolimus). These compounds are non-limiting examples of potent mTOR inhibitors the administration of which to patients can lead to mucositis. For additional information on AP23573, see WO 03/064383. For a recent reference on temserolimus (CC1779), see WO 2004/026280. For everolimus, see U.S. Pat. No. 6,384,046 and references cited therein.

2. FKBP Ligands

Compositions and methods of the present invention utilize an FKBP ligand, especially one which does not form a specific or high affinity three-part complex with FKBP and mTOR. FKBPs belong to the family of peptidylprolyl cis-trans isomerases. One of the FKBPs, FKBP-12, plays a number of important biological roles. See, e.g., Kay, 1996, Biochem J., 314, 361-385. FKBP ligands were first discovered in the form of macrolide fermentation products such as rapamycin and FK-506 and related natural products which bind to FKBP12 with high affinity. Many more FKBP ligands were subsequently discovered in the form of derivatives and analogs of those natural products, including both additional fermentation products as well as semi-synthetic compounds, i.e., synthetic derivatives of a fermentation product. For FK506 and analogous fermentation products, see, e.g., U.S. Pat. No. 4,894,366 and the more recent WO 04/78167. For analogs and derivatives of rapamycin see, e.g., WO 01/144387 and the references in Table I:

TABLE I

| | | | | |
|---|---|---|---|---|
| WO 97/10502 | WO 94/18207 | WO 93/04680 | U.S. Pat. No. 5527907 | U.S. Pat. No. 5225403 |
| WO 96/41807 | WO 94/10843 | WO 92/14737 | U.S. Pat. No. 5484799 | U.S. Pat. No. 5221625 |
| WO 96/35423 | WO 94/09010 | WO 92/05179 | U.S. Pat. No. 5457194 | U.S. Pat. No. 5210030 |
| WO 96/03430 | WO 94/04540 | U.S. Pat. No. 5604234 | U.S. Pat. No. 5457182 | U.S. Pat. No. 5208241 |
| WO 96/00282 | WO 94/02485 | U.S. Pat. No. 5597715 | U.S. Pat. No. 5362735 | U.S. Pat. No. 5200411 |
| WO 95/16691 | WO 94/02137 | U.S. Pat. No. 5583139 | U.S. Pat. No. 5324644 | U.S. Pat. No. 5198421 |
| WO 95/15328 | WO 94/02136 | U.S. Pat. No. 5563172 | U.S. Pat. No. 5318895 | U.S. Pat. No. 5147877 |
| WO 95/07468 | WO 93/25533 | U.S. Pat. No. 5561228 | U.S. Pat. No. 5310903 | U.S. Pat. No. 5140018 |
| WO 95/04738 | WO 93/18043 | U.S. Pat. No. 5561137 | U.S. Pat. No. 5310901 | U.S. Pat. No. 5116756 |
| WO 95/04060 | WO 93/13663 | U.S. Pat. No. 5541193 | U.S. Pat. No. 5258389 | U.S. Pat. No. 5109112 |
| WO 94/25022 | WO 93/11130 | U.S. Pat. No. 5541189 | U.S. Pat. No. 5252732 | U.S. Pat. No. 5093338 |
| WO 94/21644 | WO 93/10122 | U.S. Pat. No. 5534632 | U.S. Pat. No. 5247076 | U.S. Pat. No. 5091389 |

The universe of FKBP ligands was significantly enlarged by the further addition of synthetic compounds (i.e., compounds which were prepared from starting materials or intermediates that were obtained without the use of fermentation) such as those disclosed in the various documents cited below from Vertex, SmithKline Beecham, Guilford, ARIAD, etc. The practitioner thus has a large, well-defined pool of FKBP ligands to choose from in selecting a compound for use in practicing this invention.

In certain embodiments, FKBP ligands suitable for use in compositions and methods of the present invention include the N-oxalyl-pipecolyl or N-oxalyl-prolyl compounds (monomers and dimers) described in U.S. Pat. Nos. 6,133,456 and 6,150,527 and in International Patent Publication WO 97/31898. Such compounds include those of formulae I and II: 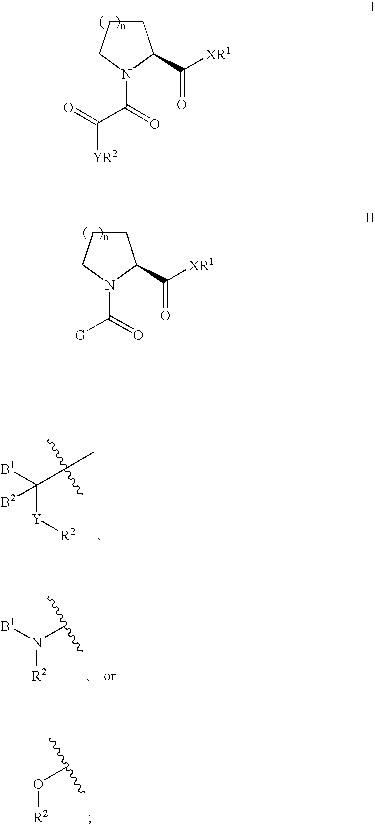
where G is selected from
n is 1 or 2;
X is O, NH or CH₂;
B¹ and B² are independently H or an alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, aryl or heteroaryl moiety, and contain up to 12 carbon atoms (not counting carbon atoms of optional substituents);
Y is O, S, NH, —NH(C═O)—, —NH(C═O)—O—, —NH(SO₂)— or NR³, or represents a covalent bond; and
R¹, R², and R³ are the same or different and are each an independently chosen alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, aryl or heteroaryl moiety; and contain up to 36 carbon atoms (not counting carbon atoms of optional substituents); and two or more of B¹, B², and R² may be covalently linked together to form a 3-7-membered ring,
   wherein each term is as described and defined in U.S. Pat. No. 6,133,456, U.S. Pat. No. 6,150,527, and in WO 97/31898.

In addition to the foregoing compounds, numerous additional compounds have been designed and/or identified as FKBP ligands. See for example, U.S. Pat. Nos. 5,192,773; 5,330,993; 5,614,547; 5,696,135; and 6,326,387; and International applications WO 92/00278; WO 92/19593; WO 94/07858; WO 96/40633; WO 96/40140; WO 96/41609; WO 98/20891; WO 98/20892; WO 98/20893 and WO 99/10340 (Vertex). See also U.S. Pat. No. 5,614,547; U.S. Pat. No. 5,786,378; U.S. Pat. No 5,795,908; U.S. Pat. No. 5,798,355; U.S. Pat. No. 5,801,187; U.S. Pat. No. 5,801,197; U.S. Pat No. 5,843,960; U.S. Pat. No. 5,846,981; U.S. Pat. No. 5,846,979; U.S. Pat. No. 5,859,031; U.S. Pat. No. 6,462,072; U.S. Pat. No. 6,509,477; WO 96/40140, WO 96/40633, WO/98/13355, WO/98/55090 and WO 01/0553 A2 (Guilford); WO 01/40185 and Dragovich et al, J Med Chem 1996, 39, 1872-1884 (Agouron); Holt et al, Bioorganic & Med Chem Letters, Vol 4, No. 2, pp 315-320; Holt et al, Bioorganic & Med Chem Letters, Vol 4, No. 2, pp 321-324; Holt et al, Bioorganic & Med Chem Letters, Vol 4, No. 2, pp 325- 328; Holt et al, J Am Chem Soc, 1993, 115, 9925-9938: U.S. Pat. No. 6,22,872 and U.S. Pat. No. 6,239,146 (BMS); and U.S. Pat. No. 6,251,932 (Astra), among many others.

For instance, Holt et al, Bioorganic & Med Chem Letters, Vol 4, No. 2, pp 315-320 discloses a variety of synthetic FKBP ligands. That paper presents a structure-activity study of those compounds as inhibitors of FKBP's activity as a peptidyl-prolyl isomerase. The paper disclosed FKBP ligands of formula I (above) in which n is 2, —XR¹ is —OEt, and —YR² is a substituted or unsubstituted aliphatic or heteroaliphatic group which may be branched, unbranched or cyclic or is aryl or alkylaryl (see Table 1 on page 316 thereof). Also disclosed are compounds in which —YR² is a branched or cyclic aliphatic group, and —XR¹, is —OEt or a more elaborate substituted or unsubstituted alkoxyl or amino group (see Table 4 on page 319). Also disclosed, among others, are compounds of formula II (above) which n is 2, and G is a substituted or unsubstituted aliphatic group, as well as potent ligands analogous to those of formula I but in which the —C(O)C(O)— group is replaced by an SO₂ group. See also Holt et al, J Am Chem Soc, 1993, 115, 9925-9938.

Additional FKBP ligands are described in U.S. Pat. No. 5,330,993. Such compounds include those of formula III: 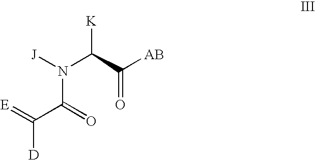
wherein A is O, NH, or N—(C₁-C₄ alkyl);
B is H, CHL-Ar, (C₁-C₆)-straight or branched alkyl, (C₁-C₆)-straight or branched alkenyl, (C₅-C₇)-cycloalkyl, (C₅-C₇)-cycloalkenyl or Ar substituted (C₁-C₆)-alkyl or alkenyl, or 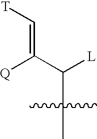
   wherein L and Q are Independently H, (C₁-C₆)-straight or branched alkyl or (C₁-C₆)-straight or branched alkenyl;
T is Ar or substituted cyclohexyl with substituents at positions 3 and 4 which are independently selected from H, OH, O—(C1-C4)-alkyl, O—(C1-C4)-alkenyl and carbonyl;
Ar is 1-naphthyl, 2-naphthyl, 2-furyl, 3-furyl, 2-thienyl, 2-pyridyl, 3-pyridyl, 4-pyridyl or phenyl having one to three substituents which are independently selected from H, halo, OH, nitro, CF₃, (C₁-C₆)-straight or branched alkyl or (C₁-C₆)-straight or branched alkenyl, O—(C₁-C₄)-straight or branched alkyl or O—(C₁-C₄)-straight or branched alkenyl, O-benzyl, O-phenyl, amino and phenyl;
D is either H or U; E is either oxygen or CH—U, provided that if D is hydrogen, then E is CH—U or if E is oxygen then D is U;
U is H, O—(C₁-C₄)-straight or branched alkyl or O—(C₁-C₄)-straight or branched alkenyl, (C₁-C₆)-straight or branched alkyl or (C₁-C₆)-straight or branched alkenyl, (C₅-C₇)-cycloalkyl or (C₅-C₇)-cycloalkenyl substituted with (C₁-C₄)-straight or branched alkyl or (C1-C4)-straight or branched alkenyl, 2-indolyl, 3-indolyl, [(C₁-C₄)-alkyl or (C₁-C₄)-alkenyl)]-Ar or Ar; and,
J is H or C₁ or C₂ alkyl or benzyl; K is (C₁-C₄)-straight or branched alkyl, benzyl or cyclohexyhethyl; or J and K may be taken together to form a 5-7 membered heterocyclic ring which may contain an oxygen (O), sulfur (S), SO or SO₂ moiety.

Additional structurally related FKBP ligands are described in WO 92/19593 and 94/07858 and elsewhere.

Further FKBP ligands are disclosed in U.S. Pat. No. 5,614,547 which are N-glyoxyl prolyl ester derivatives of formula IV: 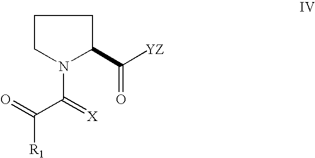
where R¹ is a C₁-C₉ straight or branched chain alkyl or alkenyl group optionally substituted with
C₃-C₈ cycloalkyl, C₃ or C₅ cycloalkyl, C₅-C₇ cycloalkenyl, Ar¹, where said alkyl, alkenyl, cycloalkyl or cycloalkenyl groups may be optionally substituted with C₁-C₄ alkyl, C₁-C₄ alkenyl, or hydroxy,
Ar¹ is 1-napthyl, 2-napthyl, 2-indolyl, 3-indolyl, 2-furyl, 3-furyl, 2-thiazolyl, 2-thienyl, 3-thienyl, 2-, 3-, 4-pyridyl, or phenyl, having one to three substituents which are independently selected from the group consisting of hydrogen, halo, hydroxyl, nitro, trifluoromethyl, C₁-C₆ straight or branched alkyl or alkenyl, C₁-C₄ alkoxy or C₁-C₄ alkenyloxy, phenoxy, benzyloxy, and amino;
X is oxygen, sulfur, CH₂, or H₂;
Y is oxygen or NR², where R² is hydrogen or C₁-C₆ alkyl; and
Z is a C₂-C₆ straight or branched, chain alkyl or alkenyl group wherein the alkyl chain is substituted in one or more positions with Ar¹ as defined above, C₃-C₈ cycloalkyl, cycloalkyl connected by a C₁-C₆ straight or unbranched alkyl or alkenyl chain, and Ar²;
Ar² is selected from the group consisting of 2-indolyl, 3-indolyl, 2-furyl, 3-furyl, 2-thiazolyl, 2-thienyl, 3-thienyl, 2-, 3-, or 4-pyridyl, and phenyl, having one to three substituents which are independently selected from the group consisting of hydrogen, halo, hydroxyl, nitro, trifluoromethyl, C₁-C₆ straight or branched alkyl or alkenyl, C₁-C₄ alkoxy or C₁-C₄ alkenyloxy, phenoxy, benzyloxy, and amino;
Z may also be the moiety 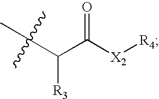
R₃ is a straight or branched alkyl C₁-C₈ group optionally substituted with C₃-C₈ cycloalkyl or Ar¹ group;
X₂ is O or NR⁵, where R⁵ is hydrogen or C₁-C₆ straight or branched alkyl or alkenyl; and
R₄ is a phenyl, benzyl, or C₁-C₅ straight or branched (optionally bearing a phenyl substituent) alkyl or alkenyl group.

These FKBP ligands share certain structural themes, but around those themes display a wide range of structural diversity. One common characteristic of such compounds is that they bind to FKBP, in some cases with very high affinity and specificity, but do not participate in any significant way in a ternary complex with FKBP and mTOR. In that sense, these compounds are are competitive inhibitors of compounds such as rapamycin and its mTOR inhibiting analogs (including, e.g., AP23573, Everolimus and Temsirolimus) and can be readily assayed as such in conventional assays of mTOR inhibiton. Yet other FKBP ligands, and dimers thereof, suitable for use in compositions and methods of the present invention include those described in U.S. Pat. No. 6,140,120.

The same is true for FK506 and its derivatives and analogs, which bind to FKBP but do not participate in the ternary complex with mTOR.

In addition to those FKBP ligands, and to FK506 and its derivatives, a variety of analogs of rapamycin, such as discussed previously, are known which bind FKBP with reasonable affinity but which, in some cases, have reduced affinity for participating in the ternary complex with FKBP and mTOR. These rapamycin analogs include, by way of non-limiting example, rapamycin derivatives with a variety of substituents at C-7, derivatization and/or epimerization at C-28, reduction of the C-24 and C-30 ketones, or a combination of such features. See e.g. the compounds in U.S. Pat. No. 6,258,823 and WO 01/14387. Such rapamycin derivatives are to be contrasted with those rapamycin derivatives described above which are potent mTOR inhibitors. To restate this, it is well known that while certain rapamycin derivatives are potent mTOR inhibitors, other rapamycin derivatives, although potent FKBP ligands, are not potent mTOR inhibitors . The literature provides many examples of these as well as a structure activity relationship to distinguish them. Thus, rapamycin analogs and derivatives that are not potent mTOR inhibitors may be used as FKBP ligands in compositions and methods of the present invention.

The FKBP ligands depicted above and described here in are merely representative examples of FKBP ligands useful in compositions and methods of the present invention. Among others these include rapamycin analogs and derivatives, typically with a molecule wight of 900-1100; FK506 and its analogs and derivatives, typically with a molecular weight of about 800-950; and synthetic small molecules, typically with a molecular weight below 700 (often between 350 and 700, unless dimerized, in which case with a molecular weight typically between about 700 and 1400).Thus, it is contemplated that any such FKBP ligand, whether synthetic (as a monomer, dimer or multimer), semi-synthetic or produced as a fermentation product, may be used in the practice of this invention. In certain embodiments, FKBP ligands for use in compositions and methods of the present invention have one or more of the following characteristics:

(i) The compound binds to FKBP12 with high affinity, i.e., it binds to human FKBP12 with an affinity at least about 0.05% that of rapamycin. In certain embodiments, it binds with an affinity at least about 0.1%, and in other embodiments at least about 1% or more, the affinity of rapamycin. Such percent affinities are designated in comparison to rapamycin using any scientifically relevant and meaningful comparative assay. Such assays include those set forth in the Examples section, infra.

(ii) The compound does not form a 3-way complex with FKBP12 and mTOR, or if it does form the complex, does so with substantially reduced affinity as compared to rapamycin. In certain embodiments, the compound forms the ternary complex with an affinity at least 10-fold lower than that of rapamycin. In other embodiments, the compound forms the ternary complex with an affinity of at least 100-fold lower than that of rapamycin. According to yet another embodiment, the compound participates in the ternary complex at least 1000-fold more weakly than does rapamycin. In still another embodiment, the FKBP ligand does not lead to measurable formation of the ternary complex. Measurement of formation of the ternary complex is achieved by methods known in the art and include those set forth in the Examples section, infra, and as described by Banaszynski, et al, J Am. Chem Soc. 2005:127, 4715- 21.

(iii) The compound is not a potent mTOR inhibitor, by which we mean that the compound has less than 10% of the mTOR inhibitory activity of rapamycin in a scientifically meaningful comparative mTOR inhibition assay. In certain embodiments, the compound has less than 5% of the mTOR inhibitory activity of rapamycin. In other embodiments, the compound has less than 1% of the mTOR inhibitory activity of rapamycin. The measurement of inhibitory activity of a compound is achieved by methods known in the art. Such methods include assays which measure a compound's ability to inhibit the proliferation of a tumor cell line sensitive to rapamycin.

Illustrative compounds which bind to FKBP12 but when bound to FKBP12 do not measurably form the further complex with mTOR (and thus have no substantial inhibitory activity against mTOR) include the compounds set forth below:

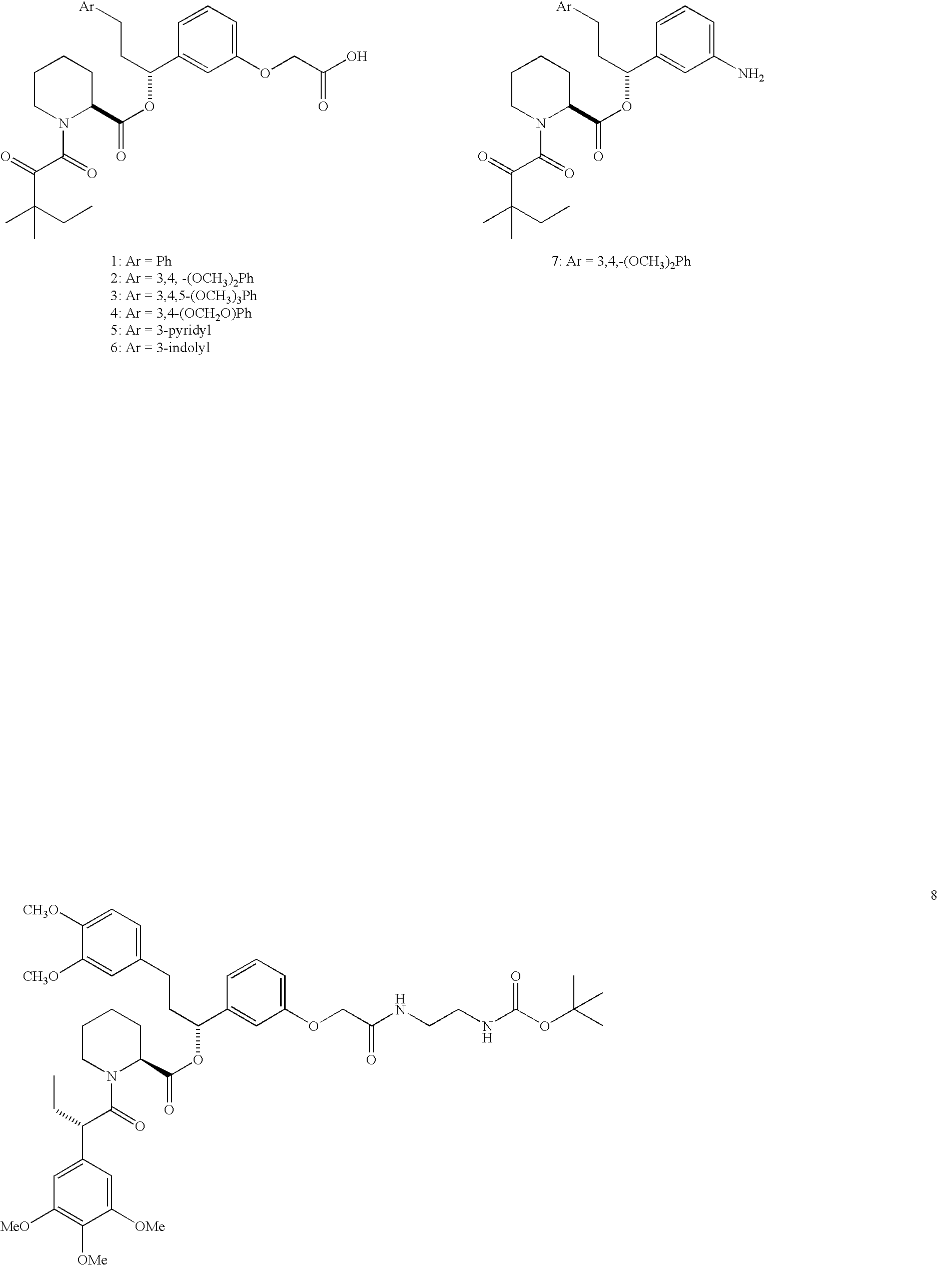

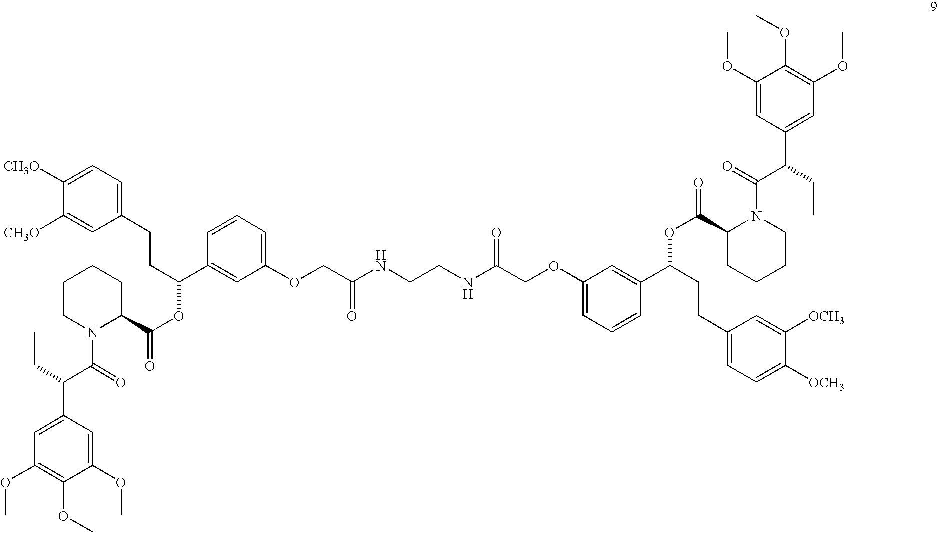

Other illustrative FKBP ligands which lack substantial mTOR binding and inhibitory activity include FK506 or FK520 and their analogs and derivatives, as well as rapamycin derivatives bearing bulky substituents such as carbamoyl, aryl, aryloxyl, heteroaryl, heteroaryloxyl, and other groups, substituted or unsubstituted, at C-7, as disclosed in U.S. Pat. No. 6,258,823.

3. Uses, Formulation and Administration

Pharmaceutically Acceptable Compositions

As discussed above, the present invention provides compositions comprising an FKBP ligand useful for the treatment of mucositis. Accordingly, in another aspect of the present invention, pharmaceutically acceptable compositions are provided, wherein these compositions comprise any such FKBP ligand as described herein, and optionally comprise a pharmaceutically acceptable carrier, adjuvant or vehicle. In certain embodiments, these compositions optionally further comprise one or more additional therapeutic agents.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or an active metabolite or residue thereof. As used herein, the term “active metabolite or residue thereof” means that a metabolite or residue thereof is also an FKBP ligand.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Basic addition salts can be prepared in situ during the final isolation and purification of the FKBP ligands by reacting a carboxylic acid-containing moiety with a suitable base such as the hydroxide, carbonate or bicarbonate of a pharmaceutically acceptable metal action or with ammonia or an organic primary, secondary or tertiary amine. Non-limiting examples of pharmaceutically acceptable salts include those based on alkali metals or alkaline earth metals such as lithium, sodium, potassium, calcium, magnesium and aluminum salts and the like and nontoxic quaternary ammonia and amine captions including ammonium, tetramethylammonium, tetraethylarnmonium, methylamine, dimethylamine, trimethylamine, triethylamine, diethylarnine, ethylamine and the like. Other representative organic amines useful for the formation of base addition salts include ethylenediamine, ethanolamine, diethanolamine, piperidine, piperazine, and the like.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Compositions of the present invention may be provided in any of a variety of formulations for administration to the patient by any of the various routes of pharmaceutical delivery. For instance, a composition may be administered, e.g., orally (in solid or liquid form), transdermal skin patch, etc. In certain embodiments, a composition of the present invention is administered orally in the form of a mouth rinse (mouthwash); an ingested solid, or liquid; or a lozenge, troche or rapidly disintegrating form. In cases of mucositis in the lower gastrointestinal tract, which may be accompanied by diarrhea, the composition may take the form of an enema. In all such cases the composition contains a therapeutically effective amount of an FKBP ligand, together with a pharmaceutically acceptable carrier or vehicle and one or more optional excipient s appropriate for the desired route and manner of administration.

The compositions of the present invention, for use in the disclosed method, can be prepared in any known or otherwise effective dosage or product form suitable for use in providing topical or local delivery of the FKBP ligands to the affected mucosa, which would include both pharmaceutical dosage forms as well as nutritional product forms suitable for use in the methods described herein

In certain embodiments, compositions are administered as oral dosage forms or products that rapidly coat or come in contact with the oral and/or esophageal mucosa, to thus provide more effective contact with the affected mucosal tissue. Suitable formulations for topical administration to oral mucosa include liquid formulations (e.g., for mouthrinse, gargle, swish, mouthwash, spray, etc.), solid dosage forms which dissolve in the mouth, and semisolid dosage forms which are applied to coat oral surfaces. Dosage or product forms of this sort include mouthwashes which the individual may swish and swallow or swish and spit out. Suitable dosage forms also include oral lozenges, tablets, gels, and other forms described herein.

Compositions and methods of the present invention are useful in any pharmaceutical or nutritional liquid product form that can directly or indirectly affect those areas of mucosa which have become or will likely develop one or more lesions following administration of an mTOR inhibitor. For example, the compositions of the present invention can be formulated in product forms to treat individuals suffering from the mucosal irritation associated with diarrhea or microbial infections such as influenza, rhino viruses, or other microbial infections that can irritate the mucosa.

Pharmaceutical compositions of the present invention are prepared by any known or otherwise effective method for formulating or manufacturing the selected product form. For example, the FKBP ligand can be formulated along with common excipients, diluents, or carriers, and formed into oral tablets, capsules, sprays, mouth washes, mouth washes, swishes, lozenges, treated substrates (e.g., oral or topical swabs, pads, or disposable, non-igestible substrate treated with the compositions of the present invention); oral liquids (e.g., suspensions, solutions, emulsions), powders, or any other suitable dosage form for topical administration to oral mucosa.

In certain embodiments, the FKBP ligand described herein are formulated as elixirs or solutions for convenient topical oral administration.

Compositions of the present invention include pharmaceutical dosage forms such as lozenges, troches or pastilles. These are typically discoid-shaped solids containing the active ingredient in a suitably flavored base. The base may be a hard sugar or sugar-free candy, glycerinated gelatin, or the combination of sugar with sufficient mucilage to give it form. Troches are placed in the mouth where they slowly dissolve, liberating the active ingredient for direct contact with the affected mucosa

Troche embodiments are prepared, for example, by adding water slowly to a mixture of the powdered active, powdered sugar, and a gum until a pliable mass is formed. A 7% acacia powder can be used to provide sufficient adhesiveness to the mass. The mass is rolled out and the troche pieces cut from the flattened mass, or the mass can be rolled into a cylinder and divided. Each cut or divided piece is shaped and allowed to dry, to thus form the troche dosage form

If the active ingredient is heat stable, or can be rendered heat stable by the use of appropriate processing precautions, it may be prepared in the form of a hard candy base. For example, sugar containing syrup can be concentrated to the point where it becomes a pliable mass. The active ingredient is then added to the mass, which is then kneaded while warm to form a homogeneous mass

The homogeneous mass is gradually worked into a pipe form having the diameter desired for the candy piece. Lozenges can be cut or sectioned off from the pipe and allowed to cool

If the active ingredient is heat labile, it may be made into a lozenge preparation by compression. For example, the granulation step in the preparation is performed in a manner similar to that used for any compressed tablet. The lozenge is made using heavy compression equipment to give a tablet that is harder than usual as it is desirable for the dosage form to dissolve or disintegrate slowly in the mouth. Ingredients are preferably selected to promote slow-dissolving characteristics

A large body of additional guidance on formulation of macrolide derivatives of rapamycin and FK506 as well as on the formulation of synthetic FKBP ligands can be obtained from the patent and scientific literature, including those documents cited herein. The FKBP ligand may also be formulated into a nutritional product as well by adaptation of the formulations of WO 03/099297.

In certain embodiments, the FKBP ligand is provided in a form suitable for topically treating the oral mucosa. Such compositions are topically administered to the oral mucosa and then swallowed or spit out. Formulation types suitable for this route of administration include liquids applied as mouth rinses; solid dosage forms that may dissolve in the mouth; and semisolids that may be applied to oral cavity surfaces.

Stability of the various FKBP ligands varies greatly with structure. However, solids for re-constitution as aqueous based solutions or suspensions prepared either by the patient or by a pharmacist prior to administration to the patient can be used, even for the less stable members of the class.

In some cases the stability of a compound in aqueous solutions is pH dependent. Procedures for choosing the optimum pH and buffering agents are well known. Other factors that affect stability in solution are also well known. For example, antioxidants may be added to reduce the rate of degradation due to oxidation.

In addition to an FKBP ligand, an aqueous liquid preparation may contain buffers, surfactants, humectants, preservatives, flavorings, stabilizers (including antioxidants), colorants, and other additives used in preparations administered into the oral cavity. In other embodiments, an FKBP ligand may be in solution or wholly or in part as a suspensions to provide liquid compositions.

In certain embodiments, compositions used as mouthwashes have a pH of about 3.5 to about 8. A pH of about 4 to about 6.5 is also contemplated. One of ordinary skill in the art would appreciate that a preparation having a pH of less than about 4 would be likely to cause a stinging sensation and preparations having a higher pH are often unpleasant to use. However, one of ordinary skill in the art would recognize that such unpleasant characteristics can be masked or otherwise made less troublesome for administration.

The preparations are buffered as necessary to provide the appropriate pH. Appropriate buffer systems can include citrate, acetate, tromethamine and benzoate systems. However, any buffer system commonly used for preparing medicinal compositions would be appropriate. Suitable vehicles include water, alcohols, glycols (polyethylene glycol or polypropylene glycol are examples), glycerin, and the like which are used to solubilize or suspend the active agent(s). Such formulations also optionally include surfactants, which include anionic, nonionic, amphoteric and cationic surfactants. Such surfactants are known in the art as appropriate ingredients for mouthwashes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Liquid formulations may contain additional components to improve the effectiveness of the product. For example, component(s) may be added to increase viscosity to provide improved retention on the surfaces of the oral cavity. Suitable viscosity increasing agents include carboxyalkyl, hydroxyalkyl, and hydroxyalkyl alkyl celluloses, xanthan gum, carageenan, alginates, pectins, guar gum, polyvinylpyrolidone, and gellan gums. High viscosity formulations may cause nausea in chemotherapy and radiation patients and are therefore not preferred. Gellan gums are sometimes preferred as viscosity modifying agents since aqueous solutions containing certain gellan gums may be prepared so that they will experience an increase in viscosity upon contact with electrolytes. Saliva contains electrolytes that may interact with such a gellan containing solution so as to increase their viscosity.

In certain embodiments, compositions of the present invention comprising an FKBP ligand are film-forming or otherwise provide a coating effect on oral mucosa. In other embodiments, compositions of the present invention are formulated to contain a mucoadhesive polymer, a viscous polymer gel or a hydrogel, by adaptation of the materials and methods of WO 2004/032843. For instance, a formulation can contain an FKBP ligand and at least one cationic polymer or a neutral polymer that becomes cationic upon contact with an aqueous medium such as saliva, thus providing a mucoadhesive or gel forming material.

The cationic polymer can be any pharmaceutically acceptable natural or synthetic polymer which has the desired physical or chemical properties to enhance retention in the mouth. Polymers will typically be cationic polymers, mucoadhesive polymers or polymers which form a gel or hydrogel that physically adheres to the mucosa. In certain embodiments, the cationic polymer is a natural polymer such as gelatin or chitosan. Most synthetic polymers including a relatively high number of carboxylic groups will be mucoadhesive. In certain embodiments, compositions of the present invention comprise one or more natural polymers. Exemplary natural polymers include zein, modified zein, casein, gelatin, gluten, chitosan, collagen, polysaccharides such as cellulose, dextrans, polyhyaluronic acid, and alginic acid. In other embodiments, compositions of the present invention comprise one or more synthetic polymers. Exemplary synthetic polymers include poly(vinyl) alcohols, polyacrylamides, polyalkylene glycols, polyalkylene oxides, polyvinyl esters, PVP, alkyl cellulose (ethyl cellulose, methyl cellulose, etc.), hydroxyalkyl cellulose (e.g. hydroxypropyl cellulose, hydroxypropyl methyl cellulose, hydroxybutyl methyl celulose, etc.), and the like. Other suitable gel or hydrogel forming polymers are well known to one of ordinary skill in the art. Other suitable polymers are biodegradable. Suitable materials and methods are as disclosed in WO 2004/032843.

Liquid bandages are well known to one of ordinary skill in the art. According to another embodiment, the present invention provides a composition in the form of a liquid bandage comprising an FKBP ligand. In certain embodiments thereof, the present invention provides a composition comprising an FKBP ligand and one or more of propylene glycol, polyvinylpyrrolidone (also known as povidone or PVP), or hyaluronic acid or a salt thereof, and optionally a flavoring agent and/or a local anaesthetic agent. In other embodiments, compositions of the present invention optionally further comprise glycyrrhetinic acid.

In certain embodiments, hyaluronic acid, or salt thereof, is present in weight percentages ranging from about 0.01 to about 5%. In other embodiments, hyaluronic acid, or salt thereof, is present in about 0.1%. According to another embodiment, glycyrrhetinic acid is present in an amount ranging from about 0.01 to 3% by weight. According to yet another embodiment, PVP is present in an amount ranging from about 1% to about 20% by weight.

Flavorings used in the mouthrinse art such as peppermint, citrus flavorings, berry flavorings, vanilla, cinnamon, and sweeteners, either natural or artificial, may be used. Flavorings that are known to increase salivary electrolyte concentrations may be added to increase the magnitude of the viscosity change. The increased viscosity will promote retention of the solutions in the oral cavity and provide greater effectiveness due to increased contact time with the affected tissues.

In order to improve the patient acceptability, it is desirable to add an appropriate coloring and/or flavoring material. Any pharmaceutically acceptable coloring or flavoring material may be used. Additionally, any of the mouthwash, mouthrinse, liquid bandage and other liquid compositions of this invention may be chilled to a temperature below body temperature, in some cases below room temperature, e.g. between 30° C. and 40° C., before administration to the patient.

Compositions of the present invention optionally further comprise one or more other active ingredients such as antibacterials, disinfectants, antifungals, analgesics, emollients, local anesthetics, and the like.

Additional antimicrobial preservatives may be component of the formulation in cases where it is necessary to inhibit microbial growth. Suitable preservatives include, but are not limited to the alkyl parabens, benzoic acid, and benzyl alcohol. The quantity of preservative may be determined by conducting standard antimicrobial preservative effectiveness tests such as that described in the United States Pharmacopoeia.

Suitable solid dosage forms include powders or tablets that are designed for constitution as solutions by dissolution or suspension in a liquid vehicle and include troches, pastilles, or lozenges that dissolve slowly in the mouth. For convenience of use, solids designed to be dissolved to prepare a liquid dosage form prior to administration are rapidly dissolving. Technologies to produce rapidly dissolving solids are well known in the art. These include spray-drying, freeze-drying, particle size reduction and optimizing the pH of the dissolution medium.

In other embodiment, the dosage form is a concentrated gel that is optionally diluted prior to administration. Such concentrated gels may be diluted with water prior to administration.

In certain embodiments, the present invention provides a unit dosage form for topical or local administration to oral mucosa comprising an FKBP ligand and optionally a pharmaceutically acceptable carrier, adjuvant or vehicle. Such unit dosage forms for use in the present invention comprise any of the formulations described above and herein for topical or local administration of an FKBP ligand to oral mucosa.

Other medicinal agents may be added for purposes of alleviating other undesirable conditions in the mouth. Such agents may include, for example, local anesthetics, anti-infective agents, and emollients. Examples of local anesthetics are lidocaine and a eutectic mixture of lidocaine and prilocaine. Lidocaine is administered in solution at a concentration of 2%, at a dose of 15 ml, at intervals of not less than three hours. The eutectic mixture is equimolar, administered at a total concentration of up to 5%. Either could be incorporated in an aerosol at similar doses.

The various compositions may include additional ingredients, such as analgesics for pain relief, antibiotics to lower the risk of or to treat infection, and other agents which might help treat mucositis or promote wound healing. Liquid compositions of the invention may further include a thickening or adhesive agent such as a mucosal-adhesive water-soluble polymer or biocompatible reverse-thermal gelation polymer to help prolong the contact of mucosa with drug, alleviate pain and/or avoid infection. In some other embodiments, the composition may include an agent which promotes cell penetration by the ligand.

Uses

In yet another aspect, a method for the treatment or prevention of mucositis induced by the systemic administration of an mTOR inhibitor in a subject is provided comprising administering an effective amount of an FKBP ligand, or a pharmaceutically acceptable composition thereof. In certain embodiments of the present invention an “effective amount” of an FKBP ligand or pharmaceutically acceptable composition thereof is that amount effective for preventing, treating or lessening the severity of mucositis induced by the systemic administration of an mTOR inhibitor or one or more symptoms thereof. The compounds and compositions, according to the method of the present invention, may be administered using any amount and any route of administration effective for preventing, treating or lessening the severity of mucositis induced by the systemic administration of an mTOR inhibitor. The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “subject”, as used herein, means an animal, preferably a mammal, and most preferably a human.

In certain embodiments, an FKBP ligand of the invention may be administered at dosage levels of about 0.01 mg/kg to about 50 mg/kg or, alternatively, from about 1 mg/kg to about 25 mg/kg, of subject body weight per day, one or more times a day, to obtain the desired therapeutic effect.

It will also be appreciated that the FKBP ligand and pharmaceutically acceptable compositions of the present invention can be employed in combination therapies, that is, the compounds and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”.

In certain embodiments, a composition of the present invention is administered to a patient prior to, concurrently with, or after administration with an mTOR inhibitor such as rapamycin, AP 23573, CCI779 or everolimus.

In practice, additional drugs and/or therapies may be administered during, prior to, or after treatment with the mTOR inhibitor and thus, during, prior to, or after the course of treatment with FKBP ligand of the present invention. Exemplary additional therapies or anticancer agents include surgery, radiotherapy (in but a few examples, gamma.-radiation, neutron beam radiotherapy, electron beam radiotherapy, proton therapy, brachytherapy, and systemic radioactive isotopes, to name a few), endocrine therapy, biologic response modifiers (e.g., interferons,), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), Gleevec™, adriamycin, dexamethasone, and cyclophosphamide. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

It is also contemplated that other agents may be administered during, prior to, or after the course of treatment with compositions of the present invention. Such agents include, without limitation: treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; immunomodulatory and immunosuppressive agents such as cyclosporin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

The contents of all cited references including literature references, issued patents, and published patent applications as cited throughout this document are hereby expressly incorporated by reference. The practice of the present invention will employ, unless otherwise indicated, conventional techniques of synthetic organic chemistry, including product recovery, purification and formulation, as well as of cell biology, cell culture, molecular biology, transgenic biology, microbiology, recombinant DNA, and immunology, which are within the skill of the art. Such techniques are explained fully in the patent and scientific literature. See, for example, in the case of biological techniques: Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Patent No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

The following examples contain additional information, exemplification and guidance which can be adapted to the practice of this invention in its various embodiments and the equivalents thereof. The examples are offered by way illustration should not be construed as limiting in any way, as many variations thereof are possible without departing from the spirit and scope of the invention. Numerous modifications and variations of the present invention should be apparent to one of skill in the art. Such modifications and variations, including design choices in selecting, preparing, formulating and administering the FKBP ligand, etc. are intended to be encompassed by the scope of the invention and of the appended claims.

The various pharmaceutical compositions of this invention may be prepared by any known or otherwise effective technique, suitable for making and formulating pharmaceutical dosage forms. Many such methods are described in the pharmaceutical arts or are otherwise well known to those skilled in their respective formulation arts

Unless otherwise specified, the active ingredient in each example is an FKBP ligand as described herein. Each example is formulated and repeated with each of compounds 1-9, disclosed above. All exemplified amounts are weight percentages based upon the total weight of the composition, unless otherwise specified.

### Biological Assays

A wide variety of FKBP binding assays and assay formats are known and are disclosed in many of the documents cited above. An illustrative assay is provided in U.S. Pat. No. 6,133,456 beginning at column 102.

Rapamycin binds to the human protein, FKBP12 and to form a tripartite complex with hFKBP12 and mTOR (also referred to previously as “FRAP”, “RAFTI” and RAPT 1”). FK506 forms a corresponding tripartite complex with hFKBP12 and calcineurin. Compounds may be characterized and compared to rapamycin with respect to their ability to bind to human FKBP12 and/or to form tripartite complexes with human FKBP12 and human mTOR (or fusion proteins or fragments containing mTOR's drug binding or “FRB” domain).

Thus, a compound's binding affinity for human FKBP12 may be determined by adaptation of known methods. For instance, the practitioner may measure the ability of a compound to compete with the binding of a known ligand to FKBP. See e.g. Sierkierka et al, 1989, Nature 341, 755-757 (test compound competes with binding of labeled FK506 derivative to FKBP). In addition, a competitive binding FP Assay is described in detail in WO99/36553 and WO96/41865. That assay permits the in vitro measurement of an IC₅₀ value for a given compound which reflects the ability of the compound to bind to an FKBP protein in competition with a labeled FKBP ligand, such as, for example, FK506.

In certain embodiment, compounds for practicing this invention include those having an IC₅₀ value in the Competitive Binding FP Assay (e.g., using a flouresceinated FK506 standard and human FKBP12) of better than about 2000 nM. In other embodiments, the compounds have an IC₅₀ value of better than about 1500 nM, or, alternatively, better than about 1000 nM. In still other embodiments, the compounds have an IC₅₀ value of better than about 500 nM.

Other compounds of particular interest bind to human FKBP12 or to a fusion protein containing such FKBP domains, with a Kd value below about 1500 nM. In other embodiments, the compounds have a Kd value below about 1250 nM, about 1000 nM, or about 750 nM, as measured by direct binding measurement (e.g. fluorescence quenching), competition binding measurement (e.g. versus FK506), inhibition of FKBP enzyme activity (rotamase), or other assay methodology.

In general, the ability of a compound to form a complex with FKBP and mTOR may be assessed from its ability to form a complex with an FKBP-fusion protein and an mTOR-fusion protein in a cell-based assay. Complex formation is assayed by measuring the occurrence of an event triggered by complex formation, i.e., multimerization or clustering of the fusion proteins. For instance, one may use cells capable of expressing DNAs encoding one fusion protein comprising one or more FKBP-12 domains (and one or more effector domains) and another fusion protein containing an FRB domain of mTOR (together with one or more effector domains). The effector domains are chosen such that they are capable, upon multimerization, of actuating a biological response. We have preferred to use cells which further contain a reporter gene under the transcriptional control of a regulatory element (i.e., promoter) which is responsive to the multimerization of the fusion proteins. The design and preparation of illustrative components and their use in so engineered cells is described in WO99/36553 and WO96/41865. (See also WO99/10510 for additional guidance on the design, assembly and delivery of nucleic acids to render cells and animals responsive to rapalogs of interest and for additional guidance on applications of such systems.) The cells are grown or maintained in culture. A compound is added to the culture medium and after a suitable incubation period (to permit gene expression and secretion, e.g. several hours or overnight) the presence of the reporter gene product is measured. Positive results, i.e., multimerization, correlates with transcription of the reporter gene as observed by the appearance of the reporter gene product. The reporter gene product may be a conveniently detectable protein (e.g. by ELISA) or may catalyze the production of a conveniently detectable product (e.g. colored). Materials and methods for producing appropriate cell lines for conducting such assays are disclosed in the international patent applications cited above in this section. Typically used target genes include by way of example SEAP, hGH, beta-galactosidase, Green Fluorescent Protein and luciferase, for which convenient assays are commercially available.

Conducting such assays permits the practitioner to select compounds possessing the desired IC₅₀ values and/or binding characteristics, i.e., compounds which bind FKBP with sufficient affinity, yet do not then strongly participate in further complex formation with mTOR.

Compounds are also readily assayed to ensure that they do not possess significant or undue mTOR inhibitory activity. mTOR inhibitory activity can be assessed using T cell proliferation assays which have been used conventionally to evaluate rapamycin analogs for their ability to inhibit proliferation of T cells. A more direct measure of mTOR inhibitory activity can be obtained using cell-based assays to measure the ability of a compound to inhibit phosphorylation of a downstream target of mTOR such as p70s6 kinase or 4E-BP1. See e.g., Peralba et al, Clinical Cancer Research Vol. 9, 2887-2892, August 2003. FKBP ligands preferred for use in this invention will optimally test negative for the ability to inhibit mTOR activity, or if some inhibitory activity is observed, it will preferably be significantly weaker than rapamycin, as discussed above.

The FKBP ligand for use in this invention should obviously be physiologically acceptable (i.e., lack undue toxicity) and can cross cellular and other membranes, as necessary for a particular application. In some cases, the compound need not have an oral bioavailability or biological half life any greater than those of rapamycin. In fact, in some embodiments reduced oral bioavailability and/or biological half life relative to rapamycin would be of interest for topical administration.

Formulation 01 for Local/Topical Delivery

The formulations may be prepared as a liquid, semi-solid, or solid containing an amount of FKBP ligand that is effective to treat or prevent mucositis. Generally, these compositions contain about 0.001 to 2 mg/ml, often 0.01 to 1 mg/ml of the compound.

Methods of Treatment

Methods of using these formulations generally involve applying the formulations topically to mucosal surfaces of the oral cavity and gastrointestinal tract. One to six or more applications are made per day beginning 24 hours before chemotherapy or radiation until conclusion of treatment or as needed thereafter.

The typical volume of a mouthwash would be between 5-15 ml of a composition containing 0.01-2.0 mg of FKBP ligand/ml of mouthwash.

A. Buffered Solutions

Solutions of compounds 1-9 are prepared by mixing each compound to a concentration of 0.1 mg/ml in a solution containing methyl and propyl parabens as antimicrobial preservatives at concentrations of 0.18% and 0.02%, respectively and tromethamine buffer.

B. Gellan Gum Buffered Formulations.

Formulations of compounds 1-9 are prepared by adding each compound to a solution containing gellan gum at a concentration of 0.5 mg/mL. The concentration of each FKBP ligand is 0.05 mg/ml. The solution also contains methyl and propyl parabens as antimicrobial preservatives at concentrations of 0.18% and 0.02%, respectively and tromethamine buffer.

C. Lower strength Gellan Gum Formulations.

Lower strength formulations of Compounds 1-9 are prepared by adding each compound to a solution containing gellan gum at a concentration of 0.5 mg/mL. The FKBP ligand concentration is 0.01 mg/mL. The solution also contains methyl and propyl parabens as antimicrobial preservatives at concentrations of 0.18% and 0.02%, respectively and tromethamine buffer.

D. Buffered Formulations.

Formulations are prepared by adding each of Compounds 1-9 to water. The suspension is added to a second solution containing a tromethamine buffer to form a mixture with a pH of approximately 8.

E. Aeorosolized FKBP Ligand.

Formulations of each of Compounds 1-9 are prepared as follows: A metered dose aerosol container is filled with the FKBP ligand and a non-FREON TI propellant. The container is equipped with a valve for delivering 500 mcg per actuation. The container is also equipped with a tube for directing the aerosol to the interior of the mouth.

F. FKBP Ligand Oral Rinse Solution.

A mixture of an FKBP ligand selected from Compounds 1-9 and buffer to promote rapid dissolution is subjected to granulation. The resultant material is dissolved in water to form an oral rinse solution containing 0.05 mg FKBP ligand/ml of formulation.

G. Effervescent Tablet Containing FKBP Ligand Formulation

Effervescent tablets are prepared of each of Compounds 1-9 with sodium bicarbonate. The tablet is dissolved in water to form an oral rinse solution containing 0.I mg/mL FKBP ligand.

H. Rapidly Disintegrating Formulations of FKBP Ligands

Rapidly disintegrating formulations of the FKBP ligands may be prepared as described in WO 2004/000223, substituting an FKBP ligand in accordance with this invention for the tetracycline compound disclosed there.

Formulation 02

In another approach, the FKBP ligand is formulated with a biocompatible reverse-thermal gelation polymer using the materials and methods of WO 02/41837.

Formulation 03

In another approach, the FKBP ligand is administered in a concentrated oral gel formulation. In this case, the FKBP ligand in an amount from 0.01 to 5 mg, usually 0.15-3 mg, of the FKBP ligand is combined with the contents of one packet of Gelclair™ (OSI Pharmaceuticals) and one tablespoon of water and stirred well. The mixture is used to rinse the mouth for at least 1 minute or as long as possible to coat the tongue, palate, throat, inside of cheeks and all oral tissue well. The material is gargled and then spit out, and administration is repeated 3 times per day or as needed, all in accordance with the normal directions for use of the Gelclair product.

Formulation 04

In another approach, the FKBP ligand, for example any of Compounds 1-9, is stirred into a glass of water to which a dose of AlkaSelzer is added, and the mixture is ingested in accordance with the normal directions for the use of alka seizer.

We claim:

1. A unit dosage form for topical or local administration to oral mucosa comprising an FKBP ligand and optionally a pharmaceutically acceptable carrier, adjuvant or vehicle.

2. The unit dosage form according to claim 1, further comprising a mucoadhesive polymer, a viscous polymer gel or a hydrogel.

3. The unit dosage form according to claim 1, wherein the FKBP ligand is a compound having one or more of the following characteristics:
(i) the compound binds to FKBP12 with high affinity, i.e., it binds to human FKBP12 with an affinity at least about 0.05% as compared to rapamycin;
(ii) the compound does not form a measurable 3-way complex with FKBP12 and mTOR; or
(iii) the compound is not a potent mTOR inhibitor.

4. The unit dosage form according to claim 3, wherein the FKBP ligand is selected from FK506 or a derivative thereof.

5. The unit dosage form according to claim 3, wherein the FKBP ligand is a rapamycin analog which binds to FKBP but which has reduced affinity for participating in the ternary complex with FKBP and mTOR.

6. The unit dosage form according to claim 3, wherein the FKBP ligand is selected from the following compounds:

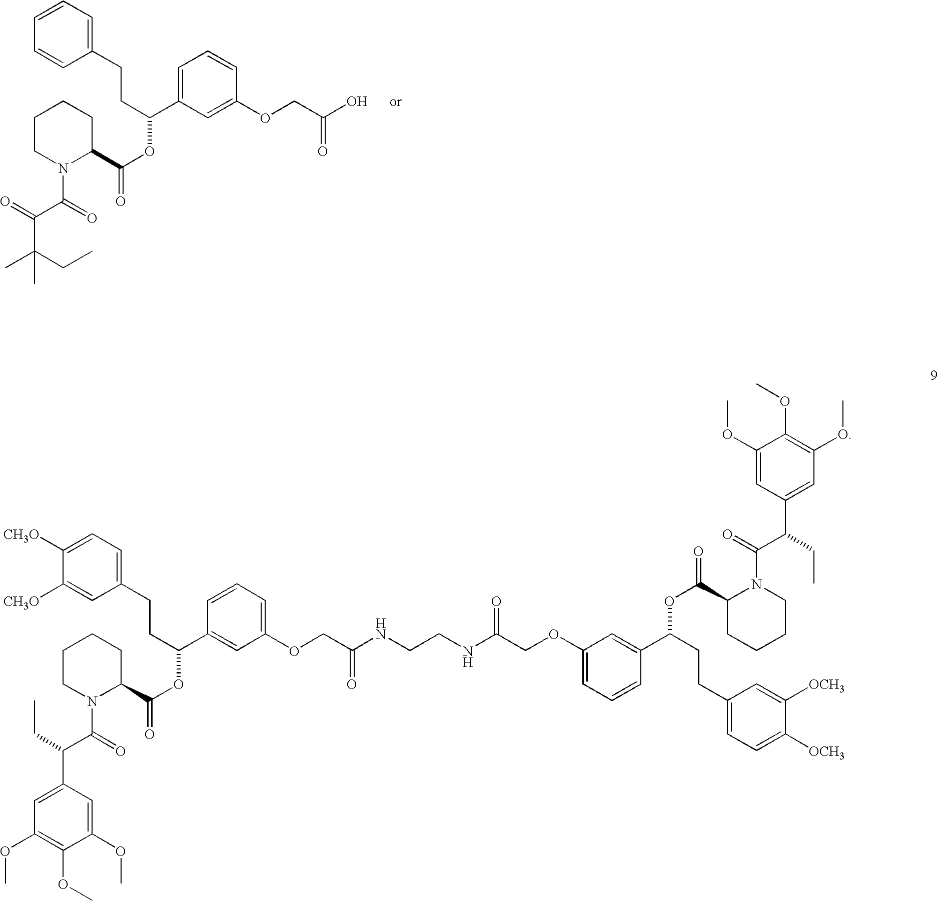

7. The unit dosage form according to claim 1, further comprising one or more active ingredients selected from antibacterials, disinfectants, antifungals, analgesics, emollients, or local anesthetics.

8. A unit dosage form of a medicament in the form of a liquid bandage, mouthwash, lozenge, troche, tablet, or pastille for topical and local administration to oral mucosa, comprising FKBP ligand and optionally a pharmaceutically acceptable carrier, adjuvant or vehicle.

9. The unit dosage form according to claim 8, further comprising a mucoadhesive polymer, a viscous polymer gel or a hydrogel.

10. The unit dosage form according to claim 9, further comprising one or more of propylene glycol, PVP, hyaluronic acid or a salt thereof, and optionally a flavoring agent.

11. The unit dosage form according to claim 8, wherein the FKBP ligand is a compound having one or more of the following characteristics:
(i) the compound binds to FKBP12 with high affinity, i.e., it binds to human FKBP12 with an affinity at least about 0.05% as compared to rapamycin;
(ii) the compound does not form a measurable 3-way complex with FKBP12 and mTOR; or
(iii) the compound is not a potent mTOR inhibitor.

12. The unit dosage form according to claim 8, wherein the FKBP ligand is selected from FK506 or a derivative thereof.

13. The unit dosage form according to claim 8, wherein the FKBP ligand is a rapamycin analog which binds to FKBP but which has reduced affinity for participating in the ternary complex with FKBP and mTOR.

14. The unit dosage form according to claim 8, wherein the FKBP ligand is a compound selected from:

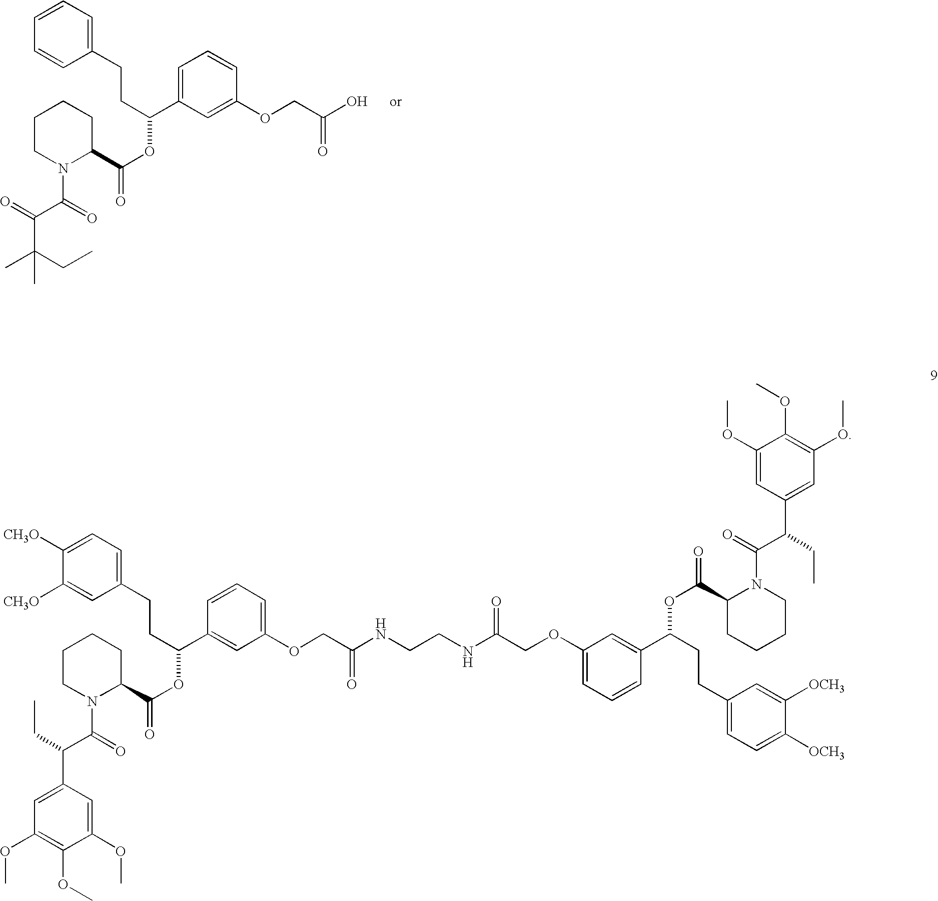

15. The unit dosage form according to claim 8, further comprising one or more active ingredients selected from antibacterials, disinfectants, antifungals, analgesics, emollients, or local anesthetics.

16. A method for treating or preventing mucositis in a patient comprising topically or locally administering to said patient an FKBP ligand, wherein the mucositis is induced by the systemic administration of an mTOR inhibitor.

17. The method according to claim 16, wherein the FKBP ligand is a compound having one or more of the following characteristics:
(i) the compound binds to FKBP12 with high affinity, i.e., it binds to human FKBP12 with an affinity at least about 0.05% as compared to rapamycin;
(ii) the compound does not form a measurable 3-way complex with FKBP12 and mTOR; or
(iii) the compound is not a potent mTOR inhibitor.

18. The method according to claim 17, wherein the FKBP ligand is selected from FK506 or a derivative thereof.

19. The method according to claim 17, wherein the FKBP ligand is a rapamycin analog which binds to FKBP but which has reduced affinity for participating in the ternary complex with FKBP and mTOR.

20. The method according to claim 17, wherein the FKBP ligand is a compound selected from: [chemical structure]
